(19) United States
(12) Patent Application Publication
(10) Pub. No.: US 2004/0230195 A1
(43) Pub. Date: Nov. 18, 2004

(54) Soft-tissue screw

(75) Inventors: Auvo Kaikkonen, Tampere (FI); Pertti Helevirta, Julkujarvi (FI); Pia Ahvenjarvi, Tampere (FI); Jukka Koljonen, Nokia (FI)

Correspondence Address: BANNER & WITCOFF, LTD., 28 STATE STREET, 28th FLOOR, BOSTON, MA, 02109-9601, US

(73) Assignee: Inion Ltd., Tampere (FI)

(21) Appl. No.: 10/438,187

(22) Filed: May 14, 2003

(51) Int. Cl. A61B 17/56

(52) U.S. Cl. 606/72

(57) ABSTRACT

A soft-tissue screw, comprising: an elongated body made of a biodegradable material and having a proximal and distal part, a channel arranged to the body in its axial direction, a first threaded part arranged to the distal part of the body, a second threaded part arranged to the proximal part of the body, a leading end arranged to the distal part, the leading end being in shape a rotationally symmetrical piece that is cut obliquely across so that a planar cut surface is created on the end, and a substantially unthreaded intermediate part arranged between the first and second threaded parts.

### FIELD OF THE INVENTION

The present invention relates to surgical devices for repairing soft-tissue tears, and more particularly to a device for repairing a meniscal tear in a knee.

### BACKGROUND OF THE INVENTION

Several methods for operating a damaged soft tissue are known. These methods include suturing and fixing the tissue by means of pin- or screw-type implants. Surgeons want to operate a torn meniscus quickly and relatively easily with an arthroscopic procedure.

The meniscus is a C-shaped cartilage positioned between the joint surfaces of the knee joint that improves the compatibility of the joint surfaces. The knee joint has two menisci. Their ends are attached to the top surface of the tibia and their sides are attached to the joint capsule of the knee joint. Tears may occur in the meniscus that hamper the functioning of the knee joint and may cause pain.

It is generally known to repair a meniscal tear arthroscopically using biodegradable implants to fasten the tear-halves together. The implants are basically either pushed, i.e. implants that fasten like pins, or screwed, i.e. screw-type implants.

Screw- or pin-type implants intended for operating soft-tissue tears are disclosed for instance in U.S. Pat. Nos. 5,569,252; 5,730,744; 6,387,113 and 6,468,277; each of which are hereby incorporated herein by reference in their entireties for all purposes.

### SUMMARY OF THE INVENTION

It is an object of the present invention to provide a bioabsorbable screw for repairing soft-tissue tears.

It is a further object of the invention to provide a bioabsorbable screw for repairing a meniscal tear in a knee.

These and other objects are attained with the screw of the present invention comprising:

an elongated body made of a biodegradable material and having a proximal and distal part,

a channel arranged to the body in its axial direction,

a first threaded part arranged to the distal part of the body,

a second threaded part arranged to the proximal part of the body,

a leading end arranged to the distal part, the leading end being in basic shape a rotationally symmetrical piece that is cut obliquely across so that a planar cut surface is created on the end, and

a substantially unthreaded intermediate part arranged between the first and second threaded parts.

The basic idea of the invention is that the obliquely cut leading end sinks easily into the tissue so that the tear being operated cannot widen. In addition, the obliquely cut leading end makes it possible to have a full height of threads in the end, which increases the pull-out force of the screw.

Further, one idea is that a substantially unthreaded intermediate part is arranged between the first and second threaded parts to reduce the friction surface of the screw and thus also the force required to install the screw into the tissue. In addition, the unthreaded intermediate part makes it possible during installation to press one side of the tear against the other by means of a cannula or a corresponding tool, which helps in pulling the tear-halves together.

The idea of an embodiment of the invention is that the threads of the first threaded part and the second threaded part differ. The profiles of the first and second threaded parts can be different from each other, for instance contrary. This improves the hold of the screw in the tissue. Further, the pitch of the first threaded part can be smaller than that of the second threaded part, which helps in pulling the tear-halves together.

Further, the idea of an embodiment of the invention is that the manufacturing material of the screw is dyed with a coloring agent, whereby the surgeon can detect the screw in the operating area easier than before.

Further, the idea of an embodiment of the invention is that at least one groove is arranged to the outer surface of the body to at least partly cut the threads of at least one threaded part. The groove helps blood vessels grow around the screw and makes it possible to also use the screw to repairing damages in the white-white area of the meniscus.

Yet another idea of an embodiment of the invention is that the second threaded part is arranged to extend to the end of the proximal part. In other words, the screw does not have a screw-head at all that could cause cartilage damage when chafing against the condylus surface.

### BRIEF DESCRIPTION OF THE DRAWINGS

In the following, the invention will be described in greater detail by means of preferred embodiments and with reference to the attached drawing, in which:

FIG. 1a is a schematic side view of a screw of the invention,

FIG. 1b is a schematic side view of the screw shown in FIG. 1a turned 90° around the center axis of the screw,

FIG. 1c is a schematic perspective view of the screw shown in FIG. 1a,

FIG. 2a is a schematic side view of a second screw of the invention,

FIG. 2b is a schematic side view of the screw shown in FIG. 2a turned 90° around the center axis of the screw,

FIG. 2c is a schematic perspective view of the screw shown in FIG. 2a,

FIG. 2d is a schematic view of the screw shown in FIG. 2a cut at the intermediate part,

FIG. 3 is a schematic perspective view of a third screw of the invention,

FIG. 4 is a schematic perspective view of a fourth screw of the invention,

FIG. 5 is a schematic perspective view of a fifth screw of the invention,

FIG. 6 is a schematic perspective view of a sixth screw of the invention.

For the sake of clarity, the invention is shown simplified in the figures. Similar parts are marked with the same reference numbers.

### DETAILED DESCRIPTION OF THE INVENTION

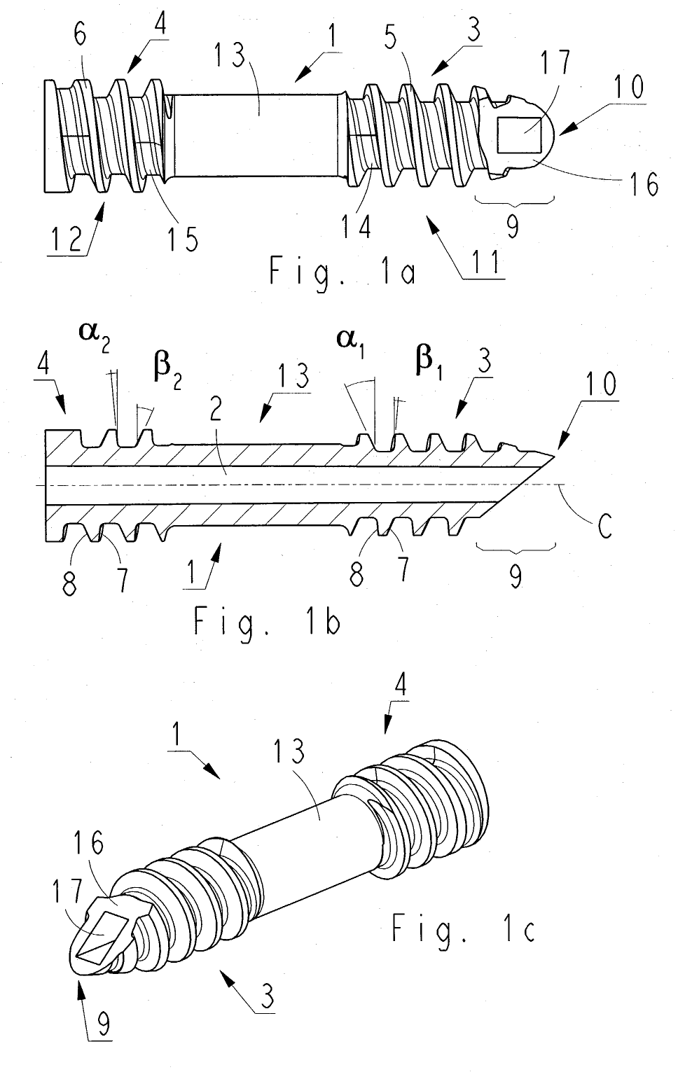

FIG. 1a is a schematic side view of a screw of the invention, FIG. 1b shows the same screw from the side and turned 90° around the center axis of the screw, and FIG. 1c is a schematic perspective view of the screw shown in FIG. 1a.

The elongated body 1 of the screw is made of a suitable biodegradable material. The body 1 has a longitudinal channel 2 having a square cross-section. A guide pin, which has a corresponding polygonal cross-section and which can thus be used to drive the screw to the tissue, can be arranged to the channel 2. The cross-section of the channel 2 can be any suitable polygon. It should be noted that the guide pin is not shown in the figure.

The distal part 11 of the screw has a first threaded part 3 and, correspondingly, the proximal part 12 of the screw has a second threaded part 4. The screw is driven into the tissue with the distal part 11 first. The pitches of the threaded parts 3, 4 are equal, but the profiles of the threads 5 and 6 are contrary to each other. In the first threaded part 3, the angle α₁ between the leading face 7 of the thread 5 and a plane perpendicular to the center axis C of the screw is bigger than the angle β₁ between the back face 8 of the thread 5 and a plane perpendicular to the center axis C of the screw. In the second threaded part 4, the angle α₂ of the leading face 7 of the thread 5 is smaller than the angle β₂ of the back face 8 of the thread 5. The angles α₁ and β₂ as well as α₂ and β₁ are now substantially equal, which is, however, not necessary. The contrary profiles of the threads 3 and 4 improve the hold of the screw.

The diameter of the core 14 of the first threaded part is somewhat smaller than the diameter of the core 15 of the second threaded part. The diameters of the cores 14, 15 of the threaded parts can also be equal, or the diameter of the core 14 of the first threaded part can be bigger than that of the core 15 of the second threaded part.

In the embodiment shown in FIGS. 1a to 1c, the diameter of the core 14, 15 of both the first and the second threaded parts 3, 4 is bigger in the section of the threaded part 3, 4 closest to the proximal part 12 than in the rest of the threaded part. However, the diameter of the core of the threaded part can also vary in some other manner or it can also be constant along the entire threaded part.

The height of the thread 5 in the first threaded part can be equal to the height of the thread 6 of the second threaded part, as in FIGS. 1a to 1c, but the heights of the threads 5, 6 can also differ from each other.

In the embodiment shown in FIGS. 1a to 1c, the pitch of the thread 5 of the first threaded part is equal to that of the thread 6 of the second threaded part. This is, however, not necessary, and the pitch of the thread 5 of the first threaded part can be smaller than the pitch of the thread 6 of the second threaded part. This type of arrangement can help pull the halves of the tear in the tissue together.

The thread 5, 6 of one or both threaded parts can also be double-helical.

An unthreaded intermediate part 13 is arranged between the first and second threaded parts 3, 4. The unthreaded intermediate part 13 helps in driving the screw into the tissue, because it reduces the friction surface between the screw and tissue. In addition, the unthreaded part 13 makes it possible to press the halves of the tear in the tissue together when the screw is being driven into the tissue. Said pressing can be done by means of a cannula or another corresponding tool.

A leading end 9 is arranged to the distal part 11 of the screw. The leading end 9 is in basic shape a circular cylinder that is obliquely cut across so that a planar cut surface 16 is formed on the end of the distal part 11 that also obliquely cuts the distal part 17 of the channel 2. The concept ‘obliquely cut’ refers herein to the fact that the cut is made obliquely in relation to the center axis C. The basic shape of the leading end 9 can also be a circular cone. The basic shape refers herein to the shape of the core 14 of the first threaded part 3.

The end 10 of the leading end 9 is offset from the center axis C. When the screw is rotated around the center axis C, the leading end 9 rotates around a circumference having a diameter that is substantially equal to the diameter of the core 14 of the first threaded part 3.

The end 10 sinks easily into the tissue and thus does not widen the tear. In addition, the structure of the leading end 9 allows the thread 5 of the first threaded part 3 to extend very close to the end 10, which increases the pull-out force of the first threaded part 3. In the screw shown in FIGS. 1a to 1c, the height of the thread 5 of the first threaded part lessens in the leading end 9, but this is not necessary, and the height of the thread 5 can be the same until the end 10.

The screw does not have a screw-head at all, but the second threaded part 4 is arranged to extend until the end of the section of the proximal part 12. This way, the screw can be driven so deep into the tissue that a screw fastened to the meniscus, for instance, does not chafe against the condylus surface.

The body 1 of the screw is made of a biodegradable polymer material absorbing into the human body that is prepared by polymerizing or copolymerizing for instance lactic acid, L-lactide, D-lactide, D,L-lactide, mesolactide, glycolic acid, glycolide or a cyclic ester copolymerized with lactide, or any other corresponding material known per se to a person skilled in the art, which will not be discussed in this context in greater detail.

The material can also be a blend, which contains a base material and one or more copolymer additives. The base material is a polymer or a copolymer of lactic acid, L-lactide, D-lactide, D,L-lactide, mesolactide, glycolic acid, glycolide or the like and optionally some other polymer or copolymer of a cyclic ester which is copolymerizable with lactide. The base material can also contain other co-monomers which impart desired properties to the material, such as α, β and γ-hydroxybutyric acid, α, β and γ-hydroxyvaleric acid and other hydroxy fatty acids (C₁₁ to C₂₅), such as stearic acid, palmitic acid, oleic acid, lauric acid and the like. Accordingly, the base material can be a polylactide, polyglycolide, poly(L-lactide), poly(D-lactide), poly(L-lactide-co-D,L-lactide), poly(L-lactide-co-mesolactide), poly(L-lactide-co-glycolide), poly(L-lactide-co-ε-caprolactone), poly(D,L-lactide-co-mesolactide), poly(D,L-lactide-co-glycolide), poly(D, L-lactide-co-ε-caprolactone), poly(mesolactide-co-glycolide), poly(mesolactide-co-ε-caprolactone) or the like. The monomer units of the copolymeric base material can be present in a ratio of 50:50 to 85:15 or in any other suitable ratio in between. For example, suitable copolymeric base materials include poly(L-lactide-co-D,L-lactide) 70:30, poly(L-lactide-co-D,L-lactide) 80:20, poly(L-lactide-co-glycolide) 85:15 and poly(L-lactide-co-glycolide) 80:20. It should be noted that the polymers and copolymers suitable for use as the base material are known per se and can be easily prepared by preparation methods which are well known to a person skilled in the art.

The copolymer additive includes one or more of lactic acid, L-lactide, D-lactide, D,L-lactide, mesolactide, glycolic acid, glycolide or the like and one or more of trimethylene carbonate and dioxanone. Certain advantageous copolymer additives include poly(L-lactide-co-trimethylene carbonate), poly(D,L-lactide-co-trimethylene carbonate), poly(mesolactide-co-trimethylene carbonate), poly(glycole-co-trimethylene carbonate), poly(L-lactide-co-dioxanone), poly(D,L-lactide-co-dioxanone), poly(mesolactide-co-dioxanone), poly(glycolide-co-dioxanone) and the like.

It should be noted that the polymers and copolymers suitable for use as the base material are known per se and can be easily prepared by preparation methods that are well known to a person skilled in the art.

The manufacturing material can be dyed with a suitable coloring agent to improve the visibility of the screw. The coloring agent can for instance be D&C Green No. 6, chemical name 1,4-bis[(4-methylphenyl)amino]-9,10-anthracenedion (CAS No. 128-80-3) or D&C Violet No. 2, chemical name 1,4-hydroxy[(4-methylphenyl)amino]-9,10-anthracenedion (CAS No. 81-48-1), both coloring agents accepted and named by FDA (Food and Drug Administration). D&C. Green No. 6 has obtained FDA approval (21CFR74.3206) for use in dyeing biodegradable sutures used in general surgery or ophthalmic surgery. D&C Violet No. 2 is approved (21CFR74.3602) for use in various sutures and biodegradable meniscus clamps made of a poly(L-lactic acid) material.

The amount of the coloring agent in the finished implant is at most approximately 0.03%, preferably 0.002 to 0.02%.

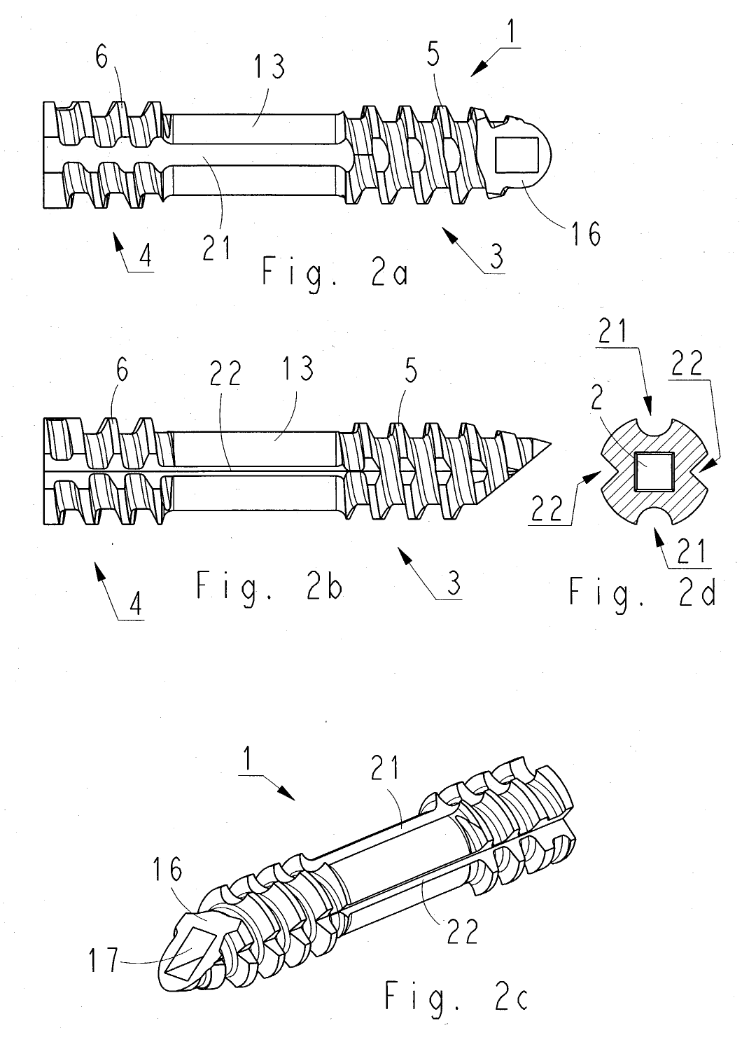

FIG. 2a is schematic side view of a screw of the invention; in FIG. 2b, the same screw is shown from the side and turned 90° around the center axis of the screw; FIG. 2c is a schematic perspective view of the screw shown in FIG. 1a; and FIG. 2d is a schematic view of the screw shown in FIG. 2a cut at the intermediate part.

The screw shown in FIGS. 2a to 2d is primarily similar to the screw shown in FIGS. 1a to 1c above, with the exception of the grooves 21 and 22 arranged on the outer surface of the screw.

The meniscus can be divided into three types of tissue: red-red with a great deal of blood vessels, red-white with fewer blood vessels than in red-red, and white-white with no blood vessels. Known implants used in repairing meniscus tears are only indicated for the operation of tears in the red-red and red-white areas. It has now been found that by arranging one or more grooves 21, 22 to the outer surface of the implant, the growth of blood vessels on the surface of the implant can be enhanced. This may allow the implant in question to be indicated for use in the white-white area in addition to the red-red and re-white areas. The implant in question thus diversifies the operating options of tears in the white-white area.

The groove 21, 22 is arranged to the body 1 so that it at least partly cuts the threads 5, 6 of the threaded parts. In the embodiment shown in FIGS. 2a to 2d, the grooves 21, 22 extend from the first threaded section 3 through the intermediate part 13 to the second threaded part 4. In the screw shown in FIGS. 2a to 2d, the grooves 21, 22 are straight and arranged axially, but they can also be arranged spirally around the body 1. Each of the grooves 21, 22 extends from one end of the screw to the other and cuts the threads 5, 6 of the first and second threaded parts and the intermediate part 13. The groove 21, 22 can also be shorter in such a manner, for instance, that it only cuts the thread of either of the threaded parts 2, 3 and possibly the intermediate part 13.

The depth of the groove 21, 22 is preferably such that it also cuts the surface of the intermediate part 13, but it can also be lower than this.

The first grooves 21 are U-shaped in cross-section, whereas the second grooves 22 are V-shaped. The cross-section of the groove 21, 22 and the number of grooves may naturally differ from the embodiment shown in FIGS. 2a to 2d.

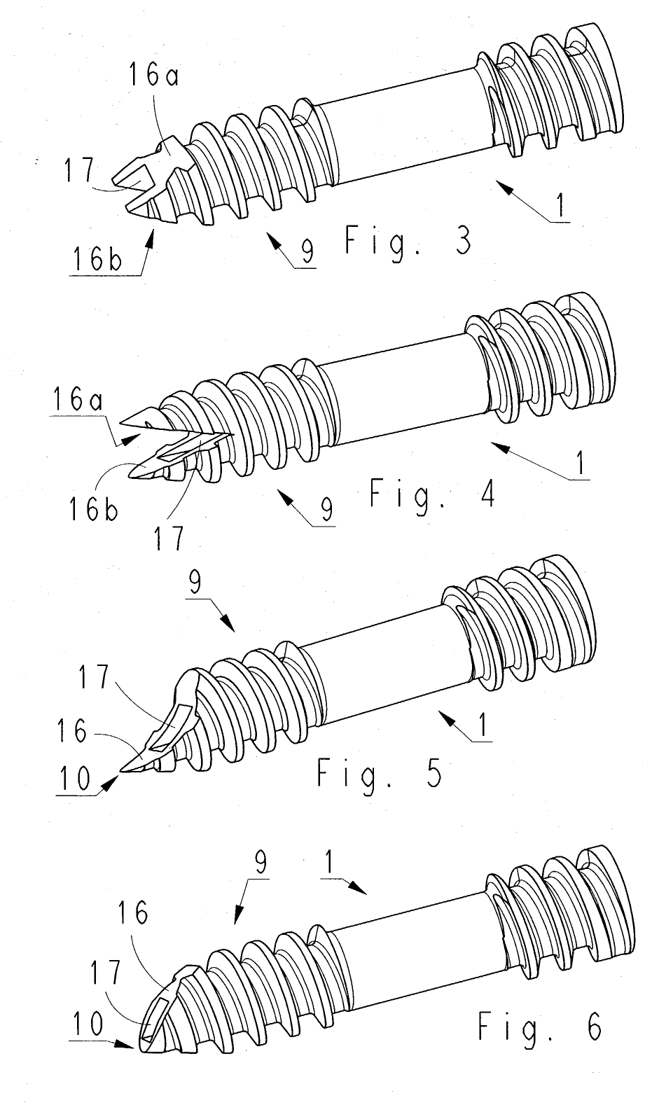

FIG. 3 is a schematic perspective view of a third screw of the invention. The structure of the screw is primarily similar to that of the screw shown in FIGS. 1a to 1c, with the exception of the leading end 9. Now, the leading end 9 is a rotationally symmetrical piece that is cut obliquely across so as to create two planar cut surfaces 16a and 16b. The cut surfaces 16a, 16b are arranged at a sharp angle to each other so that they form an edge-like end 10 in the screw. The distal end 17 of the channel divides the edge 10 into two sections.

FIG. 4 is a schematic perspective view of a fourth screw of the invention. The structure of the screw is primarily similar to that of the screw shown in FIGS. 1a to 1c, with the exception of the leading end 9. The leading end 9 now has two ends 10a, 10b arranged symmetrically in relation to the center axis of the screw and formed by cutting a V-shaped notch into the basic shape of the leading end. This way, the leading end 9 has two planar cut surfaces 16a, 16b that are oblique in relation to the axial direction of the screw. The cut surfaces 16a, 16b join on the plane running through the center axis. It should be noted that the number of ends could be higher than two. This type of leading end can have for instance four ends arranged symmetrically in relation to the center axis of the screw and the basic shape of the leading end has two V-shaped notches that cut each other perpendicularly.

FIG. 5 is a schematic perspective view of a fifth screw of the invention. Here, too, the screw structure is primarily similar to that of the screw in FIGS. 1a to 1c, with the exception of the leading end 9. Here, the planar cut surface 16 is cylindrically concave so that the end 10 becomes particularly sharp.

FIG. 6 is a schematic perspective view of a sixth screw of the invention. Here, too, the screw structure is primarily similar to that of the screw in FIGS. 1a to 1c, with the exception of the leading end 9. Here, the planar cut surface 16 is cylindrically convex so that the end 10 becomes less sharp than in the screw shown in FIGS. 1a to 1c.

The leading end 9 is designed to a cutting shape so as to facilitate the penetration of the implant into the tissue. At the same time, the leading end 9 of the implant of the invention is shaped in such a manner that the thread in it has a large surface area, which increases the pull-out force of the implant. The properties of the tissue on one hand and the pull-out force required to close the tear in the tissue on the other hand determine which shape of the leading end 9 is optimal at each time.

The term ‘planar surface’ refers herein to a surface that is formed of substantially straight surface elements arranged parallel to each other.

It will be obvious to a person skilled in the art that, as the technology advances, the inventive concept can be implemented in various ways. The invention and its embodiments are not limited to the examples described above but may vary within the scope of the claims.

1. A soft-tissue screw comprising:
an elongated body made of a biodegradable material and having a proximal and distal part,
a channel arranged to the body in its axial direction,
a first threaded part arranged to the distal part of the body,
a second threaded part arranged to the proximal part of the body,
a leading end arranged to the distal part, the leading end being in basic shape a rotationally symmetrical piece that is cut obliquely across so that a planar cut surface is created on the end, and
a substantially unthreaded intermediate part arranged between the first and second threaded parts.

2. The soft-tissue screw as claimed in claim 1, wherein
the threads of the first threaded part and the second threaded part are different.

3. The soft-tissue screw as claimed in claim 2, wherein
the pitch of the thread of the first threaded part is smaller than the pitch of the thread of the second threaded part.

4. A soft-tissue screw as claimed in claim 2, wherein
the outer diameter of the first threaded part is smaller than the outer diameter of the second threaded part.

5. A soft-tissue screw as claimed in claim 1, wherein
the leading end has one planar cut surface.

6. A soft-tissue screw as claimed in claim 1, wherein
the leading end has at least two planar cut surfaces.

7. A soft-tissue screw as claimed in claim 1, wherein
the basic shape of the leading end is a circular cylinder.

8. A soft-tissue screw as claimed in claim 1, wherein
the basic shape of the leading end is a circular cone.

9. A soft-tissue screw as claimed in claim 1, wherein
the planar cut surface is concave.

10. A soft-tissue screw as claimed in claim 1, wherein
the planar cut surface is convex.

11. A soft-tissue screw as claimed in claim 1, wherein
the planar cut surface is a straight plane surface.

12. A soft-tissue screw as claimed in claim 1, wherein
the manufacturing material is dyed with a coloring agent.

13. A soft-tissue screw as claimed in claim 12, wherein
the coloring agent is D&C Green No. 6 or D&C Violet No. 2.

14. A soft-tissue screw as claimed in claim 12, wherein
the amount of the coloring agent is at most approximately 0.03 percentage by weight.

15. A soft-tissue screw as claimed in claim 1, wherein
a groove is arranged to the outer surface of the body, to the threaded part, to at least partly cut the threads of the threaded part in question.

16. A soft-tissue screw as claimed in claim 15, wherein
the groove is arranged to extend from the first threaded part to the second threaded part.

17. A soft-tissue screw as claimed in claim 15, wherein
the groove is arranged in the direction of the center axis of the screw.

18. A soft-tissue screw as claimed in claim 1, wherein
the second threaded part is arranged to extend to the end of the proximal part.

19. A soft-tissue screw as claimed in claim 1, wherein
the screw is formed from a melt-blended polymer composition comprising:
a base material including a biodegradable polymer or copolymer, and
a copolymer additive including one or more monomers imparting a tensile strength for the implant at room temperature that is lower than a tensile strength at room temperature for an implant formed from the base material excluding the copolymer additive.

20. A soft-tissue screw as claimed in claim 1, wherein
the cross-section of the axial channel is a polygon.
